(19) United States
(12) Patent Application Publication
(10) Pub. No.: US 2002/0095692 A1
(43) Pub. Date: Jul. 18, 2002

(54) Conditional mutants of influenza virus M2 protein

(76) Inventors: David Brian Thomas, London (GB); Anita Skinner, Radlett Herts (GB); Alan James Hay, London (GB)

Correspondence Address: Kathleen Madden Williams, Palmer & Dodge, LLP, One Beacon Street, Boston, MA, 02108, US

(21) Appl. No.: 09/747,335

(22) Filed: Dec. 21, 2000

(30) Foreign Application Priority Data
Jul. 9, 1999 (GB) PCT/GB99/02204
Jun. 23, 1998 (GB) 9815040.2

(51) Int. Cl. A01K 67/00; C12N 9/00; C12N 7/00

(52) U.S. Cl. 800/8; 435/183; 435/235.1

(57) ABSTRACT

A mutant of Influenza virus M2 protein is described, which is capable of causing growth arrest in mammalian cells. Its use in a conditional cell deletion system is provided.

The present invention concerns a process for creating conditional lethal mutations in selected cells. In particular, the invention concerns means for arresting specific tissue development or destroying specific tissues in organisms in vivo.

M2, the spliced segment of the Influenza A virus matrix (M) gene, is a 97 amino acid integral membrane polypeptide containing a single membrane-spanning region. In the virus and infected cell membranes, M2 polypeptides associate into tetramers to form proton channels, thereby providing a function essential for virus replication (Holsinger, L. J. and Lamb, R. A. (1991), Virology 183: 32-43; Sugrue R. L. and Hay, A. J. (1991), Virology 180: 617-624; Pinto, L., et al. (1992), Cell 69: 517-528) by permitting proton transport in the host endosome during infection and in the trans-Golgi network during viral protein processing.

The available therapeutic agents for Influenza A virus, amantadine and rimantadine, function by blocking M2 proton channel activity (Hay, A. (1992), Seminars in Virology 3: 21-30).

Expression of M2 in certain systems, such as baculovirus or Xenopus oocytes (Schroeder et al., (1994) J Gen Virol 75:3477-3484) leads to a slow down of growth, or cell death, manifested as reduced expression of M2 protein. These adverse effects may be reversed by administration of amantadine or its analogue rimantadine. However, adverse effects have not been demonstrated in mammalian cells, which continue to grow in the presence of M2 protein.

At present, the only available conditional lethality system which may be transfected into cells is the loxP-Cre system. This combination of Cre recombinase and loxP sites, which are targeted by the recombinase, allows specific gene ablation (see Gu et al., (1994) Science 265:103).

### SUMMARY OF THE INVENTION

In a first aspect of the present invention, there is provided a mutant of Influenza virus M2 protein capable of arresting the growth of a mammalian cell.

In a second aspect, the invention relates to a transgenic non-human mammal encoding, within at least a subpopulation of the cells thereof, a transgene expressing an influenza virus M2 mutant according to the first aspect of the invention.

Preferably, the M2 mutant transgene is under tissue specific control and is thus only expressed in a certain tissue or tissues. Administration of an M2-blocking agent to the animal will prevent the growth-arresting effects of the M2 mutant protein. Arrest of tissue growth can thus be triggered by withdrawing the M2-blocking agent. This event can be timed as desired, in order to induce tissue and temporal specific arrest of cell growth. This provides an valuable tool for the study of the development of tissues.

In a third aspect, the invention relates to a method for arresting the growth of a cell comprising inserting into the cell a transgene encoding an influenza virus M2 mutant according to the first aspect of the invention.

In a fourth aspect, the invention relates to a genetic construct comprising a nucleic acid encoding an influenza M2 mutant according to the first aspect of the invention.

### DETAILED DESCRIPTION OF THE INVENTION

The present invention relates to a mutant of influenza virus M2 protein, which is defined by its ability to arrest growth of mammalian cells.

It has surprisingly been shown that cells transfected with mutants of influenza virus M2 protein are incapable of growth. The growth “arrest” observed appears not to be due to toxicity of the M2 mutants, but to arrest of the cell cycle in transfected cells, preventing further cell development and growth. Thus, the term “arrest” as used herein, refers to inhibition of cell division by at least 50% and preferably 80%, or 100% , or to the slowing of cell growth by at least 50% in the given time period and preferably 80% or 90% which ultimately results in 100% cell death. All of the above percentages are relatively to cells transfected with a control plasmid which does not encode M2 or a mutant of M2. The arrest is believed to be due to the conversion of the M2 protein from a proton channel to an ion channel, which has disadvantageous effects in mammalian cells and leads to the arrest of the cell cycle in G0 or G1. The arrest may be rescued by adding an M2-blocking agent, such as amantadine or rimantadine. These molecules, and their analogues, are capable of impeding wild-type and mutant M2 function by physically blocking the transmembrane pore which permits ion transport.

As used herein, “mutant” defines any departure from the structure of wild-type M2 protein. Thus, it includes variants in amino acid sequence as well as other derivatives, as set forth in more detail below. “M2” refers to the M2 protein of influenza A virus. In general, this term refers to M2 protein derived from any isolate of influenza A virus. Preferably, the isolate is an avian influenza virus.

Mutants of influenza virus M2 may contain amino acid deletions, additions or substitutions, subject to the requirement to maintain the ion channel activity of influenza virus M2 described herein. This includes mutations which are not in themselves responsible for the effects observed in the invention. Thus, conservative amino acid substitutions may be made substantially without altering the nature of influenza virus M2, as may truncations from the 5′ or 3′ ends. Deletions and substitutions may moreover be made to the fragments of influenza virus M2 comprised by the invention. M2 mutants may be produced from nucleic acid which has been subjected to in vitro mutagenesis resulting e.g. in an addition, exchange and/or deletion of one or more amino acids.

The fragments, mutants and other derivatives of M2 preferably retain substantial homology with the M2 sequence set forth in SEQ. ID. No. 1, taken from the Weybridge isolate of avian influenza A virus. As used herein, “homology” means that the two entities share sufficient characteristics for the skilled person to determine that they are similar in origin and function. Preferably, homology is used to refer to sequence identity. Thus, the derivatives of M2 preferably retain substantial sequence identity with SEQ. ID. No. 1 (or its encoded polypeptide product shown in SEQ. ID. No. 2).

Preferably, the sequences retain substantial homology with the coding region of SEQ. ID. No. 1, which stretches from positions 26 to 319 thereof. Advantageously, they retain substantial homology with a 25 nucleotide oligonucleotide derived from SEQ. ID. No. 1.

In an alternative embodiment, the sequences may be homologous to SEQ. ID. No. 3, which is taken from the Rostock isolate of avian influenza A virus. The M2 coding sequence in SEQ. ID. No. 3 is located between positions 1 to 26 and 715 to 982.

In a further embodiment, the M2 sequence of the invention may be homologous to a sequence selected from the group consisting of all possible sequences encoding the polypeptide of SEQ. ID. No. 2, and all possible sequences encoding the polypeptide encoded in the above-identified coding regions of SEQ. ID. No. 3.

“Substantial homology”, where homology indicates sequence identity, means more than 40% sequence identity, preferably more than 45% sequence identity and most preferably a sequence identity of 50% or more, as judged by direct sequence alignment and comparison.

Sequence homology (or identity) may moreover be determined using any suitable homology algorithm, using for example default parameters. Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http://www.ncbi.nih.gov/BLAST/blast_help.html) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994) Nature Genetics 6:119-129.

The five BLAST programs available at http://www.ncbi.nlm.nih.gov perform the following tasks:

blastp compares an amino acid query sequence against a protein sequence database;

blastn compares a nucleotide query sequence against a nucleotide sequence database;

blastx compares the six-frame conceptual translation products of a nucleotide query sequence (both strands) against a protein sequence database;

tblastn compares a protein query sequence against a nucleotide sequence database dynamically translated in all six reading frames (both strands).

tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-Periodicity internal repeats, as determined by the XNU program of Claverie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST.

The mutant provided by the present invention also includes derivatives which are amino acid mutants, glycosylation variants and other covalent derivatives of influenza virus M2 which retain the growth-arresting or cytotoxic physiological properties of M2 as set forth herein. Exemplary derivatives include molecules wherein influenza virus M2 is covalently modified by substitution, chemical, enzymatic, or other appropriate means with a moiety other than a naturally occurring amino acid.

Preferably, the invention relates to mutants derived from M2 protein isolated from avian influenza A virus (Weybridge or Rostock). Advantageously, M2 is isolated from Weybridge influenza A virus. However, the invention also relates to all variants of M2 capable of acting as proton or ion channels. Thus, also included are naturally occurring variants of influenza virus M2 found other influenza isolates. Such variants may be encoded by a related gene of the same gene family, by an allelic variant of a particular gene, a chimera of M2 genes, or represent an alternative splicing variant of an influenza virus M2 gene.

Variants which retain common structural features can be fragments of influenza virus M2. Fragments of influenza virus M2 comprise smaller polypeptides derived from therefrom. Preferably, smaller polypeptides derived from influenza virus M2 according to the invention define a single feature which is characteristic of influenza virus M2.

Fragments may in theory be almost any size, as long as they retain the ion channel activity of influenza virus M2 described herein.

The mutations responsible for endowing M2 with growth-arresting properties in mammalian cells are advantageously located in a part of the molecule which is responsible for, or associated with, the formation of a proton channel in a mammalian membrane in wild-type M2. Advantageously, the mutations are in the transmembrane domain of M2. The transmembrane domain may be defined as that part of the M2 polypeptide encoded by amino acids 26 to 43 of the Weybridge isolate M2, or equivalents thereof.

Preferably, the M2 mutants of the invention are mutated in order to endow the M2 polypeptide with ion channel activity in mammalian cells. By “ion channel activity” it is intended to denote that the activity of the M2 protein is changed from that of a proton channel, as is the case in wild-type M2, to that of a channel which allows the passage of ions other than protons. Preferably, it will allow the passage of substantially any inorganic ion, advantageously any ion.

Advantageously, mutations are effected in a residue which is involved in the formation or maintenance of the α-helical structure of the transmembrane domain. Preferably, the mutation is effected at or adjacent to position 37.

The position(s) selected for mutation are advantageously altered by amino acid substitution. Preferred substitutions are those which affect ion transport in the proton channel of M2. For example, protonation of H37 in the transmembrane domain is believed to be critical for proton channel function. Alteration of this residue, or of other residues which may affect its protonation, are expected to influence ion transport in M2.

Preferably, an Ala residue is substituted at position 37. However, other residues may be substituted at this or other positions, for example Glycine, Arginine, Glutamic acid, Glutamine or Serine. The substitution of amino acids other than Alanine at position 37 may endow the mutant with a different phenotypic characteristic to the Alanine mutant. Thus, whilst the Alanine mutant functions as an altered ion channel which allows K⁺ transport and is responsible for the arrest of the growth of the transfected cells in G1 or G0, other mutants may display different means of growth arrest and/or may be toxic to the transfected cell. When placed under the control of a tissue-specific promoter or suitable control sequence, such mutants may moreover be capable of providing conditional lethality.

Mutations may be performed by any method known to those of skill in the art. Preferred, however, is site-directed mutagenesis of a nucleic acid sequence encoding the kinase of interest. A number of methods for site-directed mutagenesis are known in the art, from methods employing single-stranded phage such as M13 to PCR-based techniques (see “PCR Protocols: A guide to methods and applications”, M. A. Innis, D. H. Gelfand, J. J. Sninsky, T. J. White (eds.). Academic Press, New York, 1990). Preferably, the commercially available Altered Site II Mutagenesis System (Promega) may be employed, according to the directions given by the manufacturer. Alternatively, the sited-directed mutagenesis method according to Kunkel et al., (1987) Enzymology 159:367 may be employed.

The M2 mutant of the invention is capable of causing growth arrest in mammalian cells. In the term “growth arrest”, all forms of growth inhibition are included. For example, the term includes toxicity, which leads to slowing of cell growth and ultimately to cell death, the inhibition of cell division and other forms of cell growth inhibition. Preferably, the term refers to the prevention of cells from proceeding through any particular phase of the cell cycle, thus preventing cell growth and division. Advantageously, growth arrested cells are arrested in phase G0 or G1 of the cell cycle.

Thus, the invention provides mammalian cells transfected with an M2 mutant according to the above aspect of the invention. Examples of useful mammalian host cell lines are epithelial or fibroblastic cell lines such as Chinese hamster ovary (CHO) cells, NIH 3T3 cells, HeLa cells or 293T cells. The host cells referred to in this disclosure comprise cells in in vitro culture as well as cells that are within a host animal.

DNA may be stably incorporated into cells or may be transiently expressed using methods known in the art. Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene to monitor transfection efficiency.

To produce such stably or transiently transfected cells, the cells should be transfected with a sufficient amount of M2-encoding nucleic acid to form M2. The precise amounts of DNA encoding M2 may be empirically determined and optimised for a particular cell and assay.

Host cells are transfected or, preferably, transformed with expression or cloning vectors as described below and cultured in conventional nutrient media modified as appropriate for inducing promoters, selecting transformants, or amplifying the genes encoding the desired sequences. Heterologous DNA may be introduced into host cells by any method known in the art, such as transfection with a vector encoding a heterologous DNA by the calcium phosphate coprecipitation technique or by electroporation. Numerous methods of transfection are known to the skilled worker in the field. Successful transfection is generally recognised when any indication of the operation of this vector occurs in the host cell. Transformation is achieved using standard techniques appropriate to the particular host cells used.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g. Sambrook et al. (1989) Molecular Cloning: A Laboratory Manual, Second Edition, Cold Spring Harbor Laboratory Press).

Transfected or transformed cells are cultured using media and culturing methods known in the art, preferably under conditions, whereby M2 encoded by the DNA is expressed.

The composition of suitable media is known to those in the art, so that they can be readily prepared. Suitable culturing media are also commercially available.

The invention moreover concerns a transgenic non-human mammal encoding, within at least a subpopulation of the cells thereof, a transgene expressing an influenza virus M2 mutant according to the preceding aspect of the invention. In the transgenic animal in question, the cells in which the mutant is expressed will be unable to grow.

Preferably, therefore, the transgene is under the control of a tissue-specific control element. This may include one or more of a tissue-specific promoter, enhancer or locus control region (LCR). Moreover, the transgene may be integrated at a specific position in the genome of the host mammal, which may provide tissue specificity as a result of the environment in which the transgene is integrated.

Transgenic animals may be generated by any suitable technique, including nuclear microinjection and the use of ES cells to produce chimeras, which are known to those skilled in the art. However, nuclear microinjection is preferred as the likelihood of transfecting all the cells of the desired tissue with the transgene is increased.

The mutants according to the invention may be regulated by the use of an M2 blocking agent. It is known that certain agents, typically amantadine, rimantadine and equivalents thereof, are capable of inhibiting the function of wild-type M2 by blocking the proton channel pore. The same agents may be used together with the mutants of the invention to block ion channel activity and thus negate the effects thereof. Thus, the growth-arrest phenotype may be rescued by administering rimantadine, amantadine or equivalents thereof to cells or transgenic animals expressing the mutants according to the invention. When the blocking agent is removed, the M2 mutant becomes operational and induces the growth arrest phenotype in cells which express it.

The invention is thus useful for the study of the development of tissues in transgenic animals, and in particular those tissues not normally accessible to manipulation. For example, the invention is applicable to the study of the tissues of the immune system.

In a further aspect, the invention concerns a genetic construct comprising a nucleic acid encoding an influenza M2 mutant according to the preceding aspects of the invention. As used herein, “genetic construct” refers to nucleic acid molecules which encode the stated constituents. In particular, the term includes discrete elements, such as vectors or plasmids, that are used to introduce heterologous DNA into cells for either expression or replication thereof. Selection and use of such vehicles are well within the skill of the artisan. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2 m plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. Thus, the vector may be suitable for integrating its DNA into the genome of the mammalian host cell.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin.

Genetic constructs according to the invention preferably contain a promoter that is recognised by the host organism and is operably linked to the M2 nucleic acid. Such a promoter may be inducible or constitutive. Preferably, the promoter is operably linked to DNA encoding M2 by removing the promoter from the source and inserting the isolated promoter sequence into the vector. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int- phage such as the CE6 phage which is commercially available (Novagen, Madison, USA). other vectors include vectors containing the lambda PL promoter such as PLEX (Invitrogen, NL), vectors containing the trc promoters such as pTrcHisXpress™ (Invitrogen) or pTrc99 (Pharmacia Biotech, SE), or vectors containing the tac promoter such as pKK223-3 (Pharmacia Biotech) or PMAL (New England Biolabs, MA., USA).

M2 gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retiovirus and Simian Virus 40 (SV40), from heterologous mammalian promoters such as the actin promoter or a very strong promoter, e.g. a ribosomal protein promoter, provided such promoters are compatible with the host cell systems. Preferably, however, a tissue-specific promoter is used. Tissue specific promoters include those specific for tissues of the immune system, including the CD2 promoter and the Lck promoter.

Transcription of a DNA encodingM2 may be increased by inserting an enhancer sequence into the vector. Enhancers are relatively orientation and position independent. Many enhancer sequences are known from mammalian genes (e.g. elastase and globin). Enhancers for use with the invention are advantageously tissue-specific, and assist in endowing the M2 expression unit with tissue specificity. The enhancer may be spliced into the vector at a position 5′ or 3′ to M2 DNA, but is preferably located at a site 5′ from the promoter.

Advantageously, a eukaryotic expression vector encoding M2 may comprise a locus control region (LCR). LCRs are capable of directing high-level integration site independent expression of transgenes integrated into host cell chromatin, which is of importance especially where the M2 gene is to be expressed in the context of a permanently-transfected eukaryotic cell line in which chromosomal integration of the vector has occurred, or in transgenic animals. In the context of the present invention, the CD2 LCR is advantageously used, for example in combination with the CD2 promoter.

Eukaryotic expression vectors will also contain sequences necessary for the termination of transcription and for stabilising the MRNA. Such sequences are commonly available from the 5′ and 3′ untranslated regions of eukaryotic or viral DNAs or cDNAs. These regions contain nucleotide segments transcribed as polyadenylated fragments in the untranslated portion of the MRNA encoding M2.

An expression vector includes any vector capable of expressing M2 nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding M2 may be inserted into a vector suitable for expression of cDNAs in mammalian cells, e.g. a CMV enhancer-based vector such as pEVRF (Matthias, et al., (1989) NAR 17, 6418).

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing M2 expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

In a still further aspect, the invention relates to a method for arresting the growth of a cell comprising inserting into the cell a transgene encoding an influenza virus M2 mutant according to the preceding aspects of the invention.

Preferably, the method includes the steps of:

(a) expressing in a cell a transgene encoding an influenza virus M2 mutant according to the invention under tissue-specific control;

(b) culturing the cells in the presence an M2 blocking agent; and

(c) culturing the cells in the absence of the blocking agent in order to induce growth arrest.

The use of blocking agents, such as amantadine or rimantadine, permits the mutant according to the invention to function as a conditional lethal agent. Administration of a blocking agent to an animal, or a patient, whose cells express the mutant according to the invention, allows the regulation of the growth of the subject cells. Therefore, the growth of selected tissues may be regulated by administration of a small molecule drug such as amantadine or rimantadine or analogues thereof.

In a further aspect of the present invention, it is a noted that rimantadine and analogues thereof are capable of crossing the blood brain barrier and the placenta. Hence, mutants according to the invention may be used to target neuronal function, by specifically ablating neural populations for the study of neurological phenomena or the treatment of neurological disorders.

The invention is described below, for the purpose of illustration only, in the following examples.

### EXAMPLE 1

Preparation of M2 H37A Mutant.

General biochemical and molecular techniques used herein are described in Sambrook et al., Molecular Cloning: A Laboratory Manual, (1989) Cold Spring Harbor, USA.

SEQ. ID. No. 1 shows the sequence of M2 protein from the Weybridge isolate of influenza A virus. His 37 of this polypeptide is changed to Ala by mutating the codon encoding position 37 from GAC to GCC by PCR using the method of Kunkel et al., (1987) Enzymology 159:367.

The mutated M2 gene is inserted in murine MEL cells (Needham et al., (1992) NAR 20:997-1003; Deisseroth et al., (1975) PNAS (USA) 72:2682-2686).

MEL cells are cultured in αAMEM medium containing 10% FCS and 200 μg/ml geneticin at 37° C. Mutant M2 protein is transiently expressed under control of the mouse β-globin promoter. Since the promoter is leaky, M2 expression occurs even in the absence of induction. Under these conditions, cells fail to grow, impeding the cloning of M2(H37A) mutants in the absence of Rimantadine.

In the presence of Rimantadine, however, the mutant can be cloned. On addition of an inducing agent (DMSO) to cells transfected with the construct there is rapid growth arrest, but in the presence of Rimantadine this is preceded by two cell doublings before terminal differentiation and growth arrest. The cell kinetics for cells expressing M2(H37A) in the presence of Rimantadine and DMSO are identical to those for cells expressing wild-type M2 in the presence of DMSO only.

### EXAMPLE 2

Construction of Lck/M2 H37A Transgene

A construct is made comprising the M2 H37A sequence under the control of the tissue specific Lck promoter.

The Lck promoter is activated during T-lymphocyte differentiation from pluripotent haematopoietic stem cells. During early thymocyte development, at the transition between CD3⁻ CD4⁻ CD8⁻ and CD3⁻ CD4⁺ CD8⁺, the Lck “proximal” promoter is switched on, and remains active until silenced at the single positive stage (CD4⁺ or CD8⁺), thereby providing a narrow window on T cell development. Previous attempts to use this promoter in conjunction with a Cre/lox gene ablation system have failed.

The Lck promoter is contained in the vector p1017, obtained from Chaffin et al., (1990) EMBO J. 9:3821-3829, which is constructed by inserting the 3.2 kb murine proximal lck promoter (Garvin et al., (1990) Int. Immunol. 2:173-180) between the EcoRI and SmaI sites of pUC19. It additionally contains a polylinker, introducing SpeI, SacII, SfiI and NotI sequences. The sequence encoding mutant M2 polypeptide is inserted at the BamHI site of p1017.

The construct is subsequently microinjected into mouse eggs and transgenic mouse lines generated.

### EXAMPLE 3

Transgenic Mice Expressing M2 H37A

All founder animals are bred to produce transgenic lines. Some hemizygous founders are then examined. The animals examined either lack a thymus with only a thymic rudiment and a small number of immature peripheral T cells, have severely reduced numbers of peripheral T cells, or develop a thymoma.

Hemizygous transgenic animals are bred to produce homozygous transgenic lines. These animals have only very small thymic rudiments.

Cells are removed from the lymphoid organs of homozygous or hemizygous transgenic animals, and control animals, and analysed by flow cytometry (FACS analysis) using anti-CD3, CD4 and CD8 antibodies stained with fluorescein isothiocyanate (FITC) or phycoerythrin (PE). The results, for homozygous transgenic animals, are shown in Table 1.

TABLE 1

| | |
|---|---|
| Control | Transgenic |

| antibody | Thymus | Spleen | Thymus | Spleen |
|---|---|---|---|---|
| CD4⁻ CD8⁻ | 4 | 50 | 95 | 95 |
| CD4⁺ CD8⁺ | 74 | — | 1 | 1 |
| CD4⁺ CD8⁻ | 16 | 33 | 4 | 4 |
| CD4⁻ CD8⁺ | 5 | 18 | 1 | — |

From the results presented in table 1, it can be seen that the cells derived from the transgenic thymus are predominantly CD4⁻ CD8⁻, suggesting an immature phenotype, whilst control animals are CD4⁺ CD8⁺. The CD4⁻ CD8⁻ cells seen in transgenic thymus tissue (95% v. 4% in control tissue) represent the earliest thymic immigrants during development of the thymus. It is believed that this is the result of the arrest of thymic tissue development at an early stage in development.

The CD4⁻ CD8⁻ cells seen in spleen are non-T cells and thus the percentage is not relevant to this analysis.

### EXAMPLE 4

Organ Culture Experiments

The transgenic mice described in example 3 have a substantially complete deletion of thymic tissue, resulting of activation of the M2 mutant under the control of the Lck promoter at an early stage in foetal development such that no early T-cells are formed and no feeders for T-cell development are present in the mouse. As a result, the lack of T-cells is not rescuable by the administration of rimantadine, since even on inactivation of the M2 mutant T-cell generation is not induced due to lack of suitable precursors.

In order to demonstrate rescue of T-cell development, organ cultures are established from 15 day old mouse embryos, substantially as follows described by Jenkinson and Anderson, (1994) Curr. Opin. Immunol. 6:293-297.

Embryo sacs are removed from 15-day pregnant mice and the embryos released by cutting the umbilical cord. The embryos are stored on ice; non-embryo tissue is discarded. Any abnormal embryos, or asynchronous embryos as judged by size, are also discarded at this stage.

Foetal thymic lobes are dissected from the embryo and cultured on Costar filters (Corning) on RPMI medium. The filters are transferred to a new well of medium every day during the experiment.

At between 7 and 10 days, the thymic lobes are harvested, transferred to an eppendorf tube containing 200 μl medium, and the cells separated by teasing out mechanically. The cells are then analysed by FACS as described above.

In a second series of experiments, the thymic rudiments are cultured in the presence of rimantadine, and the cells analysed by FACS as described above.

Culture in the presence of rimantadine is able to rescue T-cell production in the thymic rudiments. Use of an alternative promoter in the transgenic animals that would successfully arrest thymic development after the formation of early T-cells and T-cell feeders would thus result in the production of a rescuable phenotype.

1. A mutant of influenza virus M2 protein which arrests the growth of a mammalian cell.

2. The mutant protein according to claim 1 which is a mutant of the Weybridge isolate of influenza A virus.

3. The mutant according to claim 1 wherein the mutation is positioned in a residue which is part of the transmembrane domain of influenza virus M2.

4. The mutant according to claim 3 wherein the mutation is positioned at position 37.

5. The mutant according to claim 4 wherein the mutation is at His37, which is substituted with a residue selected from the group consisting of Ala, Gly, Ser, Arg and Glx.

6. A transgenic non-human mammal comprising a transgene encoding an influenza virus M2 mutant protein wherein having the characteristic of arresting cell growth, within at least a subpopulation of the cells thereof, said transgene expresses said influenza virus M2 mutant protein.

7. The transgenic animal according to claim 6 wherein the transgene is under tissue-specific control.

8. The transgenic animal according to claim 7 wherein the transgene is under the control of one or more of a tissue-specific enhancer, a tissue-specific promoter and a tissue-specific LCR.

9. The transgenic animal according to claim 6 wherein the M2 transgene causes an arrest in the growth of cells in which the protein is produced.

10. The transgenic animal according to claim 9 wherein the arrest is tissue-specific.

11. The transgenic animal according to claim 9 wherein the arrest may be prevented by administration of an M2 blocking agent to the animal.

12. A method for arresting the growth of a cell comprising inserting into the cell a transgene encoding an influenza virus M2 mutant according to claim 1.

13. The method according to claim 12 comprising the steps of:
(a) expressing in a cell a transgene encoding an influenza virus M2 mutant according to claim 1 under tissue-specific control;
(b) culturing the cells in the presence an M2 blocking agent; and
(c) culturing the cells in the absence of the blocking agent in order to induce growth arrest.

14. A genetic construct comprising a nucleic acid encoding an influenza M2 mutant according to claim 1.

15. The genetic construct according to claim 14 wherein the nucleic acid encoding the influenza virus M2 protein is operatively linked to a tissue-specific control sequence.


SEQUENCE LISTING (3 sequences)

SEQ ID NO: 1 - DNA, 339 bp, Influenza A virus
  exon (26)..(316)
agcaaaagcaggtagatgtttaaagatgagtcttctaaccgaggtcgaaacgcctaccagaaacggatgggagtgcagctgcagcgattcaagtgatcctctcgttattgccgcaagtatcattgggatcttgcactttatattgtggattcttgatcgtcttttcttcaaatgtatttatcgtcgccttaaatacggtttgaaaagagggccttctacggaaggagtgcctaagtctatgagggaagaatatcggcaggaacagcagaatgctgtggatgttgacgatggtcattttgtcaacatagagctggagtaaaaaactaccttgtttctact

SEQ ID NO: 2 - protein, 97 aa, Influenza A virus
MSLLTEVETPTRNGWECSCSDSSDPLVIAASIIGILHFILWILDRLFFKCIYRRLKYGLYRGPSTEGVPKSMREEYRQEQQNAVDVDDGHFVNIELE

SEQ ID NO: 3 - DNA, 1002 bp, Influenza A virus
  misc_feature (1)..(26): Exon
atgagtcttctaaccgaggttgaaacgtacgttctctctatcatcccatcaggccccctcaaagccgagatcgcgcagagacttgaagatgtctttgcagggaaaaacacagaccttgaggttctcatggaatggctaaagacaagaccaatcctgtcacctctgactaaagggattttggggtttgtgtttacgctcaccgtgcccagtgagcaaggactgcagcgtagacgctttgtccaaaatgccctaaatgggaatggggatccaaataacatggataaagccgtcaaactatacaggaagttgaaaagggagataacattctatggagctaaggaagtggcactcagttactctactggagcacttgccagttgtatgggcctcatatacaacagaatgggaactgtgaccacagaggtggcatttggcctagtgtgtgccacttgtgagcagattgctgattcacagcatcggtctcacagacagatggtggctaccaccaatccactaatcaggcatgagaacagaatggtaatggccagcactacagctaaggctatggagcaaatggctgggtcaattgaacaggcagcggaggccatggaggttgctagccaggctaggcagatggtgcaggcaatgaggacaattgggactcatcctagctccagtgctggtctgaaagatgatcttcttgaaaatttgcaggcctaccagaaacggatgggagtgcagatgcaacgattcaagtgaccctctcattattgccgcaagtatcattgggatcttgcacttgatattgtggattcttaatcgtcttttcttcaaatgtatttatcgtcgccttaaatacggtttgaaaagagggccttctacggaaggagtgcctgagtctatgagggaagaatatcggcaggaacagcagagtgctgtggatgttgacgatggtcattttgtcaacatagagctggagtaaaaaactaccttgtttctact